(19) United States
(12) Patent Application Publication
(10) Pub. No.: US 2026/0013511 A1
(43) Pub. Date: Jan. 15, 2026

(54) TRICHODERMA ASPERELLUM HM-8 AND APPLICATIONS THEREOF

(71) Applicant: GUO YANG INT'L MICROBIOLOGY GROUP, NEW BEACH, CA (US)

(72) Inventors: Yafei Fan, NEW BEACH, CA (US); Gen Song, NEW BEACH, CA (US); Chunguang Song, NEW BEACH, CA (US); Xiufen Zhang, NEW BEACH, CA (US); Shujian Zhang, NEW BEACH, CA (US); Yanmin Zhao, NEW BEACH, CA (US); Zhenhua Jia, NEW BEACH, CA (US); Yingchao Zhang, NEW BEACH, CA (US); Yali Huang, NEW BEACH, CA (US); Xiaolin Zhang, NEW BEACH, CA (US); Xiangyong Luo, NEW BEACH, CA (US); Hong Shen, NEW BEACH, CA (US); Qiong Song, NEW BEACH, CA (US); Zhigang Zhao, NEW BEACH, CA (US); Jirong Cui, NEW BEACH, CA (US); Yanhong Wu, NEW BEACH, CA (US); Lili Han, NEW BEACH, CA (US); Dingxin Guo, NEW BEACH, CA (US)

(21) Appl. No.: 19/239,258

(22) Filed: Jun. 16, 2025

(30) Foreign Application Priority Data
Jul. 9, 2024 (CN) 202410915842.3

(51) Int. Cl. A01N 63/38; A01P 1/00; A01P 3/00; C12N 1/14

(52) U.S. Cl. CPC A01N 63/38; A01P 1/00; A01P 3/00; C12N 1/145

(57) ABSTRACT

A Trichoderma asperellum HM-8 of the invention has the dual ability to antagonize Erwinia and Fusarium solani f. sp. batatas. The microbial agent prepared from the Trichoderma asperellum HM-8 strain can efficiently control sweet potato root rot and ginger stem basal rot. The spore count of Trichoderma asperellum HM-8 is not less than 108 CFU/g.

### 1. TECHNICAL FIELD

The present invention relates to the technical field of agricultural microorganisms, and in particular to a Trichoderma asperellum HM-8 and applications thereof.

### 2. BACKGROUND ART

Sweet potato root rot is a soil-borne fungal disease that seriously harms sweet potato production, occurring in both the seedbed and field stages. After sweet potatoes are infected with root rot, the root and stem bases become black and rotten, resulting in few and small tubers or no tuber formation, leading to a decline in the yield and quality of sweet potato tubers. The main pathogen of sweet potato root rot is Fusarium solani (Mart.) Sacc. f. sp. batatas Mc Clure, belonging to the Deuteromycota subphylum, also known as Fusarium solani f. sp. batatas. Infected soil and disease residues in the soil are the main primary infection sources for the following year. The pathogens of sweet potato root rot are transmitted through the soil, with the highest density in the plough layer and the most severe disease occurrence. The pathogens in the soil can survive for at least 3 years, and their vertical distribution can reach a soil layer of 100 cm, although the highest density is in the plough layer.

Ginger stem basal rot, also known as ginger soft rot or ginger stem rot, has extremely complex causes. Reported pathogens include the bacterium Ralstonia solanacearum, Erwinia, and the fungi Pythium myriotylum and Fusarium oxysporum. Studies have shown that in some ginger-growing areas in Hebei, the main pathogen causing ginger stem basal rot is Erwinia, which mainly exists in the soil.

At present, the main measures for controlling sweet potato root rot and ginger stem basal rot in production include agricultural control, chemical agents control, and biological control. Although chemical agents are widely used in agricultural production due to their many advantages, with the gradual enhancement of human health and environmental protection awareness, the long-term and extensive improper use of pesticides has gradually revealed many problems, such as environmental pollution, pesticide residues, the development of resistance, and direct toxicity to non-target organisms. Biological control has to some extent compensated for the shortcomings of chemical pesticides and will play a significant role in the sustainable development of agriculture in the future.

### 3. Summary of the Invention

The object of the invention is to provide a Trichoderma asperellum HM-8 capable of effectively controlling sweet potato root rot and ginger stem basal rot.

The invention adopts the following technical scheme:
A Trichoderma asperellum HM-8, with the preservation number of CGMCC No. 41051, was preserved in the General Microbiology Center of the China General Microbiological Culture Collection Center (address: Beijing, China) on Jan. 23, 2024.

Further, the Trichoderma asperellum HM-8 can antagonize Fusarium solani f. sp. batatas.

Further, the Trichoderma asperellum HM-8 can antagonize Erwinia.

Further, the Trichoderma asperellum HM-8 has the dual ability to antagonize both Erwinia and Fusarium solani f. sp. batatas.

A microbial agent comprising bacterial cells, spores, metabolites, fermentation broth, and/or fermentation products of the above-mentioned Trichoderma asperellum HM-8.

In the microbial agent, the spore count of Trichoderma asperellum HM-8 is not less than 10⁸ CFU/g.

The microbial agent further contains soluble starch.

An application of the above-mentioned Trichoderma asperellum HM-8 in biological control.

An application of the above-mentioned Trichoderma asperellum HM-8 in the biological control of sweet potato root rot.

An application of the above-mentioned Trichoderma asperellum HM-8 in the biological control of ginger stem basal rot.

The beneficial effects of the present invention are as follows: the Trichoderma asperellum HM-8 of the invention has the dual ability to antagonize Erwinia and Fusarium solani f. sp. batatas. The microbial agent prepared from the Trichoderma asperellum HM-8 strain can efficiently control sweet potato root rot and ginger stem basal rot, with control effects reaching 88.09% and 80.20%, respectively, and significantly increasing the yields of sweet potatoes and ginger. In comparison, its control effect and yield are significantly higher than those of chemical pesticides, making it possible to replace pesticides for disease control and reduce environmental pollution.

### 4. Brief Description of Accompany Drawings

FIG. 1 shows the inhibition rate test of strain HMC03 against Fusarium solani f. sp. batatas.

FIG. 2 shows the inhibition rate test of strain HMC04 against Fusarium solani f. sp. batatas.

FIG. 3 shows the petri dish inhibition effect of the fermentation broth of strain HMC03 against Erwinia.

FIG. 4 shows the petri dish inhibition effect of the fermentation broth of strain HMC04 against Erwinia.

FIG. 5 shows the bacterial colony morphology of Trichoderma asperellum HM-8.

FIG. 6 shows the morphology of Trichoderma asperellum HM-8 stained with cotton blue under a microscope.

FIG. 7 shows a phylogenetic tree of Trichoderma asperellum HM-8.

### 5. Specific Embodiment of the Invention

The invention will be further described in detail below with reference to specific embodiments. It should be noted that the specific embodiments are illustrative of the present invention rather than limiting.

### Embodiment 1 Screening of Strains

Soil samples were collected from the rhizosphere soil of ginger plants without ginger stem basal rot in a severely affected plot of a ginger plantation in Liushouying Town, Funing District, Qinhuangdao City, Hebei Province. A total of 6 samples were collected, each weighing 100 g, and the 6 soil samples were uniformly mixed. Ten grams of the mixed soil sample was placed in an Erlenmeyer flask containing 90 mL of sterile water and shaken thoroughly. The soil dilution and spread plate method was used for treatment. After gradient dilution of the soil sample, 100 μL of dilutions at 10⁴, 10⁵, and 10⁸ were evenly spread on plates containing PDA medium, and cultured upside down at 25° C. in the dark. After 2 days, observation was carried out daily. When mycelia grew out, mycelial blocks were picked and transferred to new PDA plates for continued culture. When spores were produced, single-spore isolation and purification were performed. The purified strains were numbered and stored on slants in a 4° C. refrigerator for later use.

The formula of the above-mentioned PDA medium is as follows: potato 200 g/L, agar 20 g/L, glucose 20 g/L. The preparation method is as follows: peel and cut potatoes, boil for 30 min, filter through gauze, add sucrose and agar, heat and stir until the agar is completely dissolved, make up to 1000 mL, dispense, and sterilize at 121° C. for 20 min for later use.

The agar plate confrontation assays was used to determine the antagonistic effect of the above-numbered strains against Fusarium solani f. sp. batatas. The specific method was as follows: (1) the above-purified and numbered strains were cultured on PDA medium; (2) after 4 days, a 5-mm-diameter bacterial disc of Fusarium solani f. sp. batatas was punched with a 200 μL pipette tip and inoculated in a middle of a PDA medium plate; (3) bacterial discs of the isolated and purified numbered strains were punched and symmetrically inoculated on the above PDA medium plate at a distance of 3 cm from the middle of the PDA medium plate, and cultured at 28° C. for 5-7 days, with 3 replicates for each treatment. Observation was carried out daily. When the screened strain grew full on the plate, the colony diameter of Fusarium solani f. sp. batatas was measured, and the inhibition rate of the screened strain against Fusarium solani f. sp. batatas was calculated.

$$Inhibition rate ( \% ) = ( 9 - colony diameter of Fusarium solani f . sp . batatas ) \div 9 \times 100.$$

In the formula, 9 is the diameter of the plate (cm).

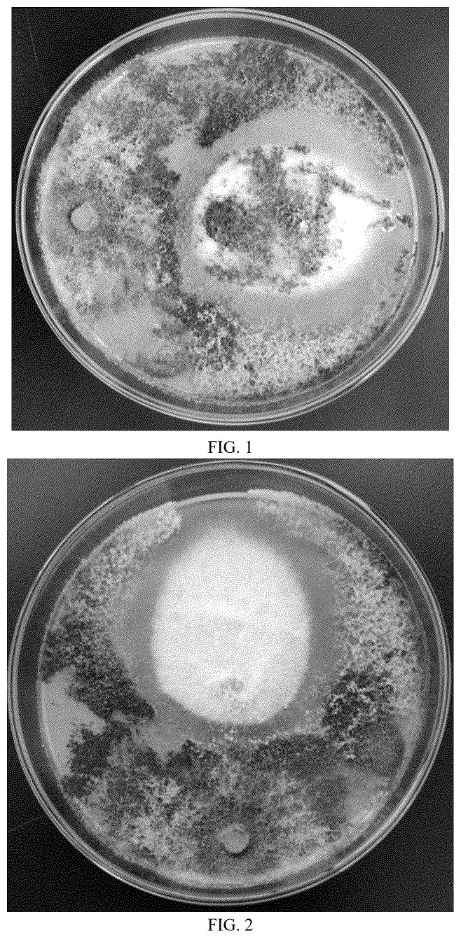

The detection showed that strains numbered HMC03 and HMC04 had higher inhibition rates against Fusarium solani f. sp. batatas, reaching 100% and 53.33%, respectively, as shown in FIG. 1, FIG. 2, and Table 1.

TABLE 1
Inhibition Rates of HMC03 and HMC04 against
Fusarium solani f. sp. Batatas.

| Strain number | HMC03 | HMC04 |
|---|---|---|
| Average diameter of colony diameter | 0 | 4.2 |
| of Fusarium solani f. sp. batatas | | |
| Inhibition rate (%) | 100 | 53.33 |

Antagonism test of strains HMC03 and HMC04 against Erwinia. To detect the antibacterial activity of the fermentation broths of HMC03 and HMC04, the specific steps were as follows:
1) prepare a bacterial suspension of the pathogen Erwinia with a concentration of 10′CFU/mL; pipette 1 mL of the Erwinia fermentation broth onto the surface of an LB medium plate, and spread the bacterial suspension evenly with a spreader.
2) punch holes with a diameter of 5 mm in the above medium with a 1000 μL pipette tip;
add the fermentation broth of the strain HMC03 or HMC04 to be detected into the holes, filling them without overflowing;
incubate at 37° C. for 3 days, observe the results, measure the diameter of the inhibition zone with a vernier caliper, and repeat the experiment 3 times.
judgment criteria: an inhibition zone diameter ≥15 mm indicates that Erwinia is extremely sensitive to the fermentation broth of strain HMC03 or HMC04; 10 mm≤inhibition zone diameter≤15 mm indicates moderate sensitivity; 6 mm≤inhibition zone diameter≤10 mm indicates low sensitivity, and no inhibition zone indicates insensitivity.

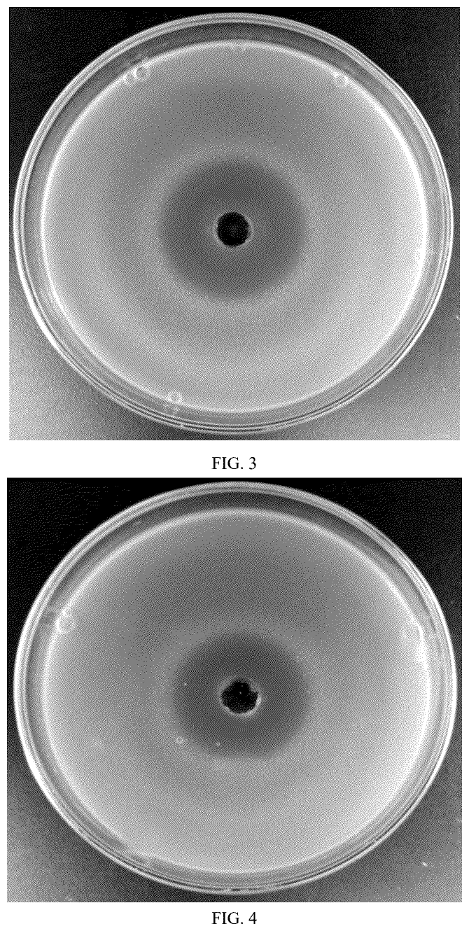

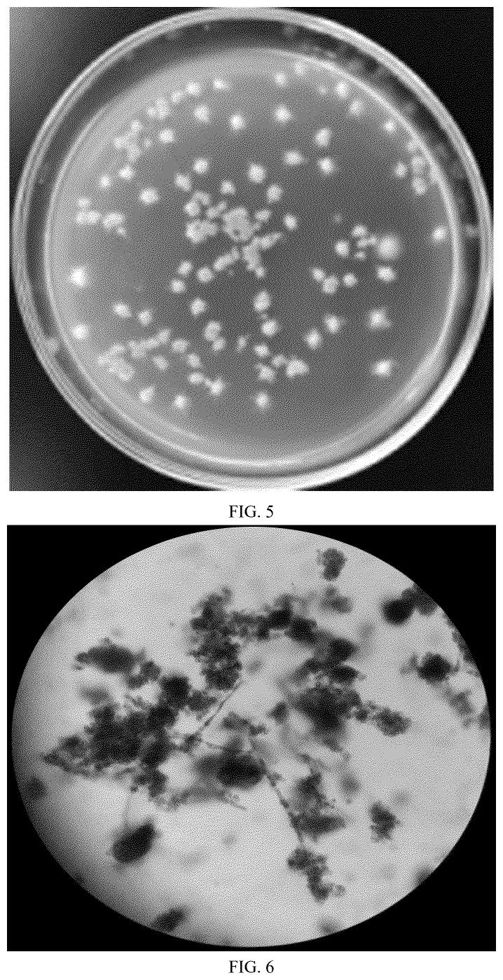

The detection results showed that the average diameter of the inhibition zone produced by the fermentation broth of strain HMC03 against Erwinia was 30.4 mm (FIG. 3), and the average diameter of the inhibition zone produced by the fermentation broth of strain HMC04 against Erwinia was 24.6 mm (FIG. 4), among which the fermentation broth of strain HMC03 had a stronger inhibitory ability against Erwinia.

### Embodiment 2 Identification of Strain HMC03

The isolated strain HMC03 was inoculated onto PDA medium, the colony of strain HMC03 was white, round, without obvious concentric rings or radial morphology at the initial culture on the PDA plate, and could grow full on a 9 cm petri dish after 72 hours of culture on the PDA medium, with a fast growth rate. After 5 days, green conidia were produced from middle to periphery, then covering the entire plate. Its aerial hyphae were dense, the surface was tight like a blanket, and the edge was white and flocculent. Microscopic observation showed that the conidia of HMC03 were spherical, preliminarily proving that HMC03 was a mould. As shown in FIG. 5 and FIG. 6.

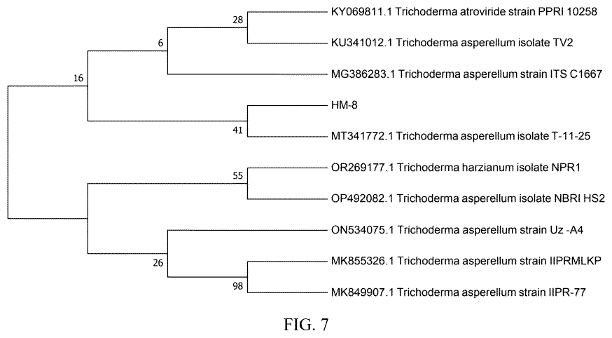

The strain HMC03 was entrusted to the Institute of Biology, Hebei Academy of Sciences for species identification. With reference to “6.5” in NY/T 1736-2009, the ITS region (as shown in SEQ ID No. 1) of the strain was amplified by PCR, the amplified product was recovered and sequenced, the sequence was compared with the sequences registered in the GenBank database for homology, and a phylogenetic tree was constructed using MEGA7.0 as shown in FIG. 7. The ITS region sequence of the strain had 100% homology with Trichoderma asperellum.

Based on sequence similarity, phylogenetic analysis, and morphological identification, the strain HMC03 was identified as Trichoderma asperellum, named HM-8. The strain was preserved in the General Microbiology Center of the China Microbial Culture Collection Management Committee (address: No. 3, Beichen West Road, Chaoyang District, Beijing, China) on Jan. 23, 2024, with the biological preservation number CGMCC No. 41051.

### Embodiment 3 Preparation of Strain HM-8 Microbial Agent

Strain HM-8 was inoculated into PDA medium and cultured in an incubator at 25° C. for 7 days. Three to four bacterial blocks were taken from it and added to a seed bottle. The seed solution was prepared by shaking culture at 25° C. and 180 rpm for 48 hours for later use.

The seed solution was added to a seed fermentation tank at a volume ratio of 1:150 (the composition of the seed fermentation tank medium: by weight, containing 2% sucrose, 1% peptone, 0.2% NaCl, 0.01% K₂HPO₄, 0.01% MgSO₄, 0.5% CaCO₃) for culture, and then added to a chlamydospore fermentation medium fermentation tank at a volume ratio of 1:20 (chlamydospore fermentation medium: by weight, containing 2.5% starch, 1.5% yeast powder, 5.0% corn steep liquor, 0.4% CaCO₃, 0.01% ZnSO₄, 0.03% MgSO₄). Culturing was carried out at 28° C. for 132 hours until 90% of the hyphae formed chlamydospores to obtain chlamydospore fermentation broth, which was then freeze-spray dried. The chlamydospore dry powder of the strain was mixed with soluble starch to form a wettable powder. Detection showed that the agent contained 1.2×10⁸ CFU/g of spores of Trichoderma asperellum HM-8. The obtained wettable powder was the HM-8 microbial agent used in the following embodiments.

### Embodiment 4 Application of HM-8 Microbial Agent in the Control of Ginger Stem Basal Rot

The experiment was conducted in a ginger plantation in Funing District, Qinhuangdao City, Hebei Province, which had continuous cropping of ginger for many years and severe continuous cropping diseases (stem basal rot). A 20 m×6 m area was selected as one experimental area, with 10 ridges of ginger planted in each experimental area, approximately 500±30 plants, and the variety was Mianjiang (Zingiber striolatum). A total of 12 experimental areas were set up, with protective rows established between each experimental area. Three experimental areas out of the 12 were randomly selected for each treatment group. The experimental design was as follows:
(1) blank control group 1: plant ginger in the ginger furrows without applying any bacterial powder.
(2) control group 2: use 4 kg/mu of 50% Carbendazim wettable powder mixed with 20 kg/mu of soil, evenly spread in the ginger furrows, and then plant ginger in the ginger furrows. The 50% Carbendazim wettable powder was purchased from the market.

Bacterial powder treatment groups: first, evenly spread the bacterial powder of the HM-8 microbial agent wettable powder prepared in Embodiment 3 of the present invention in different amounts in the ginger furrows, and then plant ginger in the ginger furrows. Among them: treatment group 1: the dosage of the HM-8 microbial agent was 1 kg/mu, mixed with 20 kg/mu of moist soil; treatment group 2: the dosage of the HM-8 microbial agent was 3 kg/mu, mixed with 20 kg/mu of moist soil.

During the growth period of ginger, other cultivation and management measures were carried out in accordance with local production technical specifications. At the time of ginger harvest, the incidence of ginger stem basal rot was counted, and the control efficiency of the Trichoderma asperellum HM-8 microbial agent against ginger stem basal rot was calculated. The specific results are shown in Table 2.

TABLE 2
Control Effect of Trichoderma asperellum
HM-8 Microbial agent on Ginger Stem Basal Rot

| | | | Yield(kg) of |
|---|---|---|---|
| | Disease | Control | experimental area |
| Treatment | index(%) | efficiency(%) | (20 m × 6 m) |
| Blank control group 1 | 72.40 | — | 493.3 |
| Control group 2 | 15.73 | 78.27 | 683.5 |
| Treatment group 1 | 11.08 | 84.70 | 721.5 |
| Treatment group 2 | 8.62 | 88.09 | 757.4 |

Disease grading criteria: grade 0, healthy ginger plants without disease; grade 1, local disease on mother ginger plants, with healthy offspring ginger plants; grade 2, offspring ginger plants with disease spots but no withering; grade 3, partial withering of ginger clumps (30%-50%); grade 4, basic withering or complete withering of ginger clumps, with less than 60% discoloration and rot of ginger flesh; grade 5, complete withering of ginger clumps, with more than 60% rot of ginger flesh.

The calculation formulas for disease index and relative control effect are respectively:

$$disease index ( \% ) = [ \sum ( number of diseased plants at each grade \times corresponding disease grade ) / ( total number of surveyed plants \times highest disease grade ) ] \times 100 ; \\ relative control effect ( \% ) = [ ( disease index of control group - disease index of treatment group ) / disease index of control group ] \times 100.$$

As can be seen from the results in Table 2, compared with the blank control group, each treatment group could significantly reduce the disease index of ginger stem basal rot, among which the control efficiency of treatment group 2 was as high as 88.09%, and the yield increased by 53.5% compared with the blank control group; compared with control group 2, each treatment group also had a better control effect, among which treatment group 2 had a control effect nearly 10% higher than that of control group 2, and the yield increased by 10.8%. This indicates that the Trichoderma asperellum HM-8 microbial agent provided by the invention has a significant control effect on ginger stem basal rot, can replace pesticides, and reduce environmental pollution.

### Embodiment 5 Application of HM-8 Microbial Agent in the Control of Sweet Potato Root Rot

The test was conducted in a sweet potato plantation of a grower in Lulong Town, Lulong County, Hebei Province. The test site was a hillside field in a hilly area, with loam soil, medium fertility conditions, and poor water conservancy conditions. Except for the water used for planting, it basically relied on natural precipitation. The test site had continuously planted sweet potatoes for 10 years, and root rot had occurred seriously in the past 3 years, with a yield loss of approximately 30%.

The experiment set a total of 3 treatments, as shown in Table 3.

TABLE 3
Application Test Scheme of HM-8 Microbial
Agent in Controlling Sweet Potato Root Rot

| Treatment | Treatment content |
|---|---|
| Treatment 1 | Soak sweet potato seedlings in 100-fold solution of HM-8 |
| | microbial agent for 10 minutes + soil treatment with HM-8 |
| | microbial inoculant (3 kg microbial powder mixed with 21 |
| | kg moist soil per mu, applied by hole, 6 g per hole). |
| Treatment 2 | Soak sweet potato seedlings in 200-fold solution of 50% |
| | Carbendazim wettable powder for 10 minutes + mix 4 kg of |
| | 50% Tuijunte (a fungicide containing Thiram and Ziram) |
| | wettable powder with 20 kg of soil per mu, and apply 6 g |
| | per hole before sweet potato transplanting. |
| Control (CK) | No pesticide application, soak sweet potato seedlings in |
| | clear water for 10 minutes. |

Among them, 50% of Carbendazim wettable powder and 50% Tuijunte wettable powder were both purchased from the market.

Each treatment was repeated 3 times. In the test plots, 5 rows of sweet potatoes were planted, with a row spacing of 0.8 m, a row length of 10 m, and a plant spacing of 0.2 m. The plots were randomly arranged, with each plot covering an area of approximately 48 square meters. The tested sweet potato variety was ‘Tengfei’. All plots were uniformly pre-treated against nematodes and underground pests, using 35% phoxim microcapsule agent (for controlling underground pests) and 10% fosthiazate granules (for controlling nematodes). All tested sweet potatoes were planted on May 10, 2023. On October 11, the diagonal fixed 5-point survey method was used, with 10 plants surveyed per treatment to measure and record sweet potato yield and diseased tubers (tubers with individual fresh weight below 50 g were not included in the statistics). The survey statistics are shown in Table 4.

TABLE 4
Control Effect of Each Treatment on Sweet
Potato Root Rot and Sweet Potato Yield

| | | | Yield(kg) of |
|---|---|---|---|
| | Disease | Control | experimental area |
| Treatment | index(%) | efficiency(%) | (20 m × 6 m) |
| Treatment 1 | 14.74 | 80.20 | 196.85 |
| Treatment 2 | 18.77 | 74.79 | 180.60 |
| Control (CK) | 74.45 | — | 67.40 |

The severity of sweet potato root rot was graded according to the disease symptoms of tubers, divided into 5 grades in total:
Grade 0: normal tubers without symptoms;
Grade 1: individual roots turn black (number of diseased roots less than 10% of the total), no lesions on underground stems, no obvious impact on tuber formation;
Grade 2: a small number of roots turn black (number of diseased roots accounting for 10%-25% of the total), individual lesions on underground stems and tubers, mild impact on tuber formation;
Grade 3: nearly half of the roots turn black (number of diseased roots accounting for 25.1%-50.0% of the total), multiple lesions on underground stems and tubers, significant impact on tuber formation, with woody roots;
Grade 4: most roots turn black (number of diseased roots accounting for more than 50% of the total), numerous and large lesions on underground stems, no tuber formation or even death.

The diseased tuber rate, disease index, and control effect were calculated using the following formulas:

$$disease tuber rate ( \% ) = ( number of diseased tubers in the plot / number of harvested tubers in the plot ) \times 100. \\ disease index ( \% ) = [ \sum ( number of tubers at each disease grade \times corresponding grade representative value ) / ( number of harvested tubers in the plot \times highest disease grade ) ] \times 100. \\ control effect ( \% ) = [ ( disease index of blank control plot - disease index of chemical treatment plot ) / disease index of blank control plot ] \times 100.$$

The results in Table 4 show that both Treatment 1 and Treatment 2 had good control effects and yield-increasing effects on sweet potato root rot. Among them, the control effect of the former reached over 80.20% compared with the control, and the plot yield was also 129.45 kg higher than the control; compared with Treatment 2, Treatment 1 also had better control effects and yield-increasing effects, which indicates that the Trichoderma asperellum HM-8 microbial agent of the present invention also has a significant control effect on sweet potato root rot, can replace pesticides, and reduce environmental pollution.

The above are only preferred embodiments of the invention and are not intended to limit the invention. Any modifications, equivalent substitutions, improvements, etc. made within the spirit and principles of the present invention shall be included in the protection scope of the present invention.

1. A Trichoderma asperellum HM-8, with the preservation number of CGMCC No. 41051.

2. A microbial agent, comprising bacterial cells and/or spores of the Trichoderma asperellum HM-8 according to claim 1.

3. The microbial agent according to claim 2, wherein the spore count of Trichoderma asperellum HM-8 is not less than 10⁸ CFU/g.

4. The microbial agent according to claim 2, wherein the microbial agent further contains soluble starch.

5. An application of the Trichoderma asperellum HM-8 according to claim 1 in the biological control of sweet potato root rot.
